(19) United States
(12) Patent Application Publication
(10) Pub. No.: US 2013/0225904 A1
(43) Pub. Date: Aug. 29, 2013

(54) SYSTEM AND METHOD FOR MAGNETIC CONTROL OF AN ANESTHETIC

(71) Applicant: University of Virginia, (US)

(72) Inventors: George T. Gillies, Charlottesville, VA (US); Robert H. Thiele, Charlottesville, VA (US)

(73) Assignee: UNIVERSITY OF VIRGINIA, Charlottesville, VA (US)

(21) Appl. No.: 13/780,207

(22) Filed: Feb. 28, 2013

Related U.S. Application Data
(60) Provisional application No. 61/605,062, filed on Feb. 29, 2012.

(51) Int. Cl. A61N 2/00

(52) U.S. Cl. CPC A61N 2/002
USPC 600/12

(57) ABSTRACT

Systems and methods for magnetic control of an anesthetic or analgesic agent are disclosed. In an exemplary embodiment, the method includes delivering a fluidic agent to a spinal region of a subject. The fluidic agent includes an anesthetic and/or analgesic component and a magnetic component. The method also includes applying a magnetic field to an area proximate a target area of the spinal region such as to maintain the fluidic agent at the target area, move the fluidic agent towards the target area, or move the fluidic agent away from the target area.

### CROSS-REFERENCE TO RELATED PATENT APPLICATION

This application claims priority to and the benefit of, pursuant to 35 U.S.C. §119(e), U.S. provisional patent application Ser. No. 61/605,062, filed Feb. 28, 2012, entitled “System and Method for Manipulation of Hyperbaric Lidocaine Using Magnetic Field,” by George T. Gillies and Robert H. Thiele, the contents of which is incorporated herein in its entirety by reference.

Some references, which may include patents, patent applications, and various publications, are cited in a reference list and discussed in the disclosure provided herein. The citation and/or discussion of such references is provided merely to clarify the description of the present disclosure and is not an admission that any such reference is “prior art” to any aspects of the present disclosure described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [4] represents the 4th reference cited in the reference list, namely, Kawashita et al., Preparation of Ferrimagnetic Magnetite Microspheres for in Situ Hyperthermic Treatment of Cancer. Biomaterials 2005; 26:2231-8.

### BACKGROUND

Spinal anesthesia relies on injection of local anesthetic and adjuvant drugs into the intrathecal space. To control the spread of intrathecal drugs, dextrose is often added to the solution, rendering it hyperbaric. Generally, hyperbaric mixtures move toward the gravitationally dependent portions of the spine. If, for anatomical or patient positioning reasons, the anesthetic drugs reach the T1-4 cardioaccelerator fibers, profound bradycardia, hypotension, and sometimes cardiac arrest may result. The incidence of cardiac arrest after spinal blockade may be as high as 6.4 per 10,000 anesthetics, and cardiac arrest was the primary damaging event in 38% and 32% of non-obstetric and obstetric neuraxial anesthetic claims, respectively, from 1980 to 1999. ([1],[2]). Anesthesiologists currently influence block height by modifying the dose of anesthetic drugs and by changing the angle between the patient's back and the surface of the earth, thereby enabling gravitational forces to concentrate the drug in the dependent region of the spinal canal. Gravity may also be used in an attempt to produce a unilateral block. However, in certain instances, gravitational forces alone may not be sufficient to control block height.

It is with respect to these and other considerations that the various embodiments described below are presented.

### SUMMARY

In one aspect, the present invention relates to a method that, in an exemplary embodiment, includes delivering a fluidic agent to a spinal region of a subject. The fluidic agent includes an anesthetic and/or analgesic component and a magnetic component. The method also includes applying a magnetic field to an area proximate a target area of the spinal region such as to maintain the fluidic agent at the target area, move the fluidic agent towards the target area, or move the fluidic agent away from the target area.

In another aspect, the present invention relates to a system. In an exemplary embodiment, the system includes a fluid delivery device that is configured to deliver a fluidic agent to a spinal region of a subject. The fluidic agent includes an anesthetic and/or analgesic component and a magnetic component. The system also includes at least one magnet that is configured to apply a magnetic field to an area proximate a target area of the spinal region such as to maintain the fluidic agent at the target area, move the fluidic agent towards the target area, or move the fluidic agent away from the target area.

In yet another aspect, the present invention relates to a computer-readable storage medium with stored instructions that, when executed by one or more processors, cause a computer to perform specific functions. In an exemplary embodiment, the functions include causing at least one magnet to apply a magnetic field to an area that is proximate a target area of a spinal region of a subject, such as to maintain a fluidic agent at the target area, move the fluidic agent towards the target area, or move the fluidic agent away from the target area. The fluidic agent includes an anesthetic component and/or analgesic component and a magnetic component.

Other aspects and features of embodiments of the present invention will become apparent to those of ordinary skill in the art, upon reviewing the following detailed description in conjunction with the accompanying figures.

### BRIEF DESCRIPTION OF THE DRAWINGS

FIGS. 1A and 1B are diagrams illustrating a system 100 for magnetic control of a fluid agent 104 in a spinal region R of a subject S, according to an exemplary embodiment of the present invention.

FIGS. 2A-2D provide a sequence 200 of time-lapse illustrations of the spread of a magnetic, hyperbaric local anesthetic solution 206 in the absence of an applied magnetic field, according to an exemplary embodiment of the present invention.

FIGS. 3A-3D provide a sequence 300 of time-lapse illustrations of a magnetic, hyperbaric local anesthetic solution 206 delivered to a target area 208 and maintained in the target area 208 by an externally-applied magnetic field from a magnetic source device 302, according to an exemplary embodiment of the present invention.

FIG. 4 is a flow diagram illustrating operational steps of a method 400 for magnetic control of a fluid agent in a spinal region of a subject, according to an exemplary embodiment of the present invention.

FIG. 5 is a computer architecture diagram showing illustrative computer hardware architecture for a computing system 500 capable of implementing aspects of the present invention according to exemplary embodiments disclosed herein.

### DETAILED DESCRIPTION

Although exemplary embodiments of the present invention are explained in detail, it is to be understood that other embodiments are contemplated. Accordingly, it is not intended that the present invention be limited in its scope to the details of construction and arrangement of components set forth in the following description or illustrated in the drawings. The present invention is capable of other embodiments and of being practiced or carried out in various ways.

It must also be noted that, as used in the specification and the appended claims, the singular forms “a,” “an” and “the” include plural referents unless the context clearly dictates otherwise.

In describing exemplary embodiments, terminology will be resorted to for the sake of clarity. It is intended that each term contemplates its broadest meaning as understood by those skilled in the art and includes all technical equivalents that operate in a similar manner to accomplish a similar purpose.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Ranges may be expressed herein as from “about” or “approximately” one particular value and/or to “about” or “approximately” another particular value. When such a range is expressed, another embodiment includes from the one particular value and/or to the other particular value. As used herein, “about” means within 20 percent or closer of a given value or range.

As discussed herein, a “subject” or “patient” may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g. rat, dog, pig, monkey), etc. It should be appreciated that the subject may be any applicable human patient, for example.

It is also to be understood that the mention of one or more steps of a method does not preclude the presence of additional method steps or intervening method steps between those steps expressly identified. Method steps may be performed in a different order than those described herein. Similarly, it is also to be understood that the mention of one or more components in a device or system does not preclude the presence of additional components or intervening components between those components expressly identified.

The following detailed description is directed to systems and methods for magnetic control of an anesthetic. In the following detailed description, references are made to the accompanying drawings that form a part hereof and that show, by way of illustration, specific embodiments or examples. In referring to the drawings, like numerals represent like elements throughout the several figures.

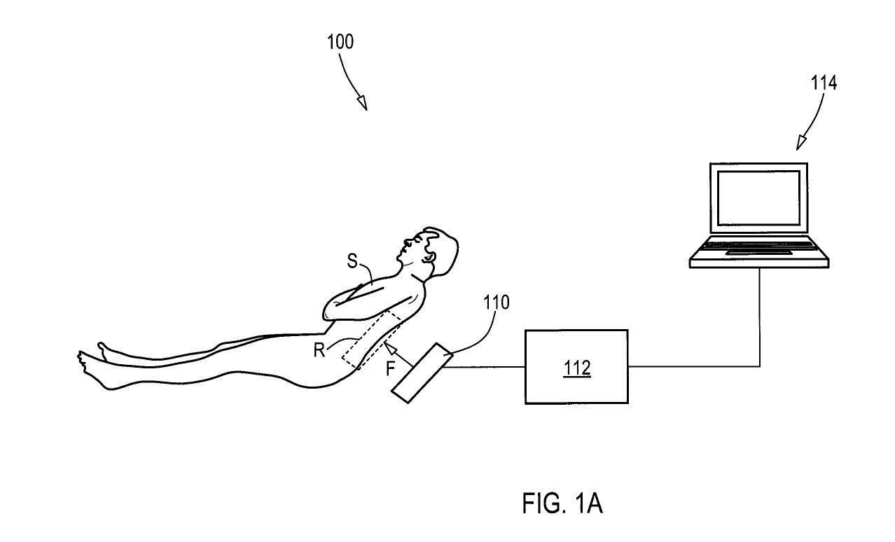

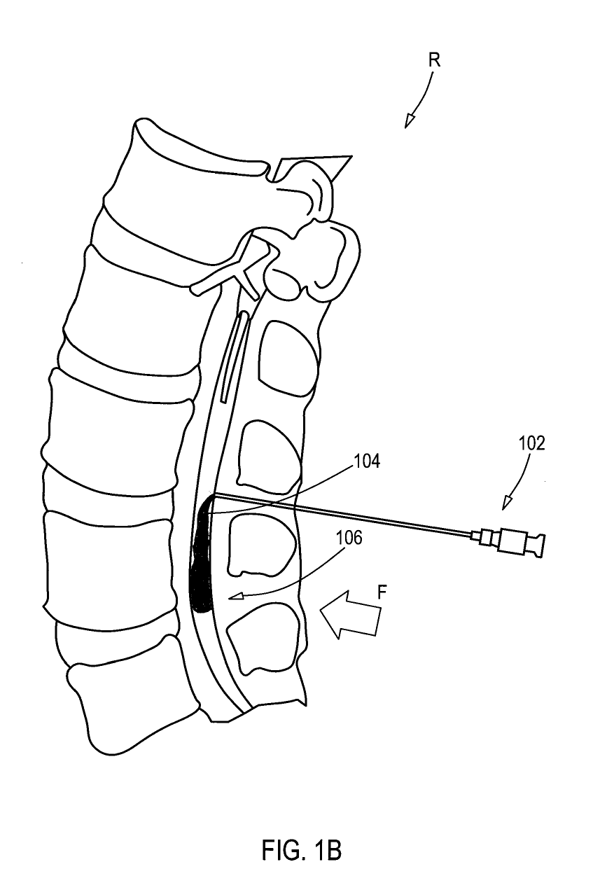

FIGS. 1A and 1B illustrate a system 100 according to an exemplary embodiment of the present invention. As shown in FIG. 1B, the system 100 includes a fluid delivery device 102 with a syringe and introducer needle or cannula for delivering a fluidic agent 104 to a target area 106 of a spinal region R of a living human subject S. The target area 106 corresponds to an intrathecal space of the spinal canal of the subject S. The fluidic agent 104 includes a magnetic component with a suspension of particles having permanent and/or induced magnetic moments. The magnetic component can include a ferrofluid, for example. The fluidic agent 104 also includes an anesthetic component and/or analgesic component, for example a local anesthetic such as lidocaine, bupivacaine, or ropivacine and/or an analgesic agent such as morphine or fentanyl. A magnetic source device 110 for generating a magnetic field F includes one or more magnets and is configured to apply the magnetic field F to an area of the spinal region R that is proximate the target area 106, in order to maintain the fluidic agent 104 in the target area 106, move the fluidic agent 104 towards the target area 106, or move the fluidic agent 104 away from the target area 106. The entire bolus of the fluidic agent 104 acts in response to the effects of the magnetic field as it couples to the magnetic component of the fluidic agent 104.

The magnetic source device 110 can include one or a combination of sources for generating a static magnetic field. For example, ferromagnetic, paramagnetic, diamagnetic, or ferrimagnetic sources may be used. One or more permanent magnets can be included, for example samarium-cobalt or neodymium-boron-iron magnets. Additionally or alternatively, electromagnets can be used, for example electromagnets with magnetic gradient coils and associated amplifiers. As shown in FIG. 1A, the magnetic source device 110 is coupled to a controller 112 and a user computer 114. The controller 112 may include a real-time control sequencer configured to control the magnetic field F by monitoring and manipulating field parameters such that the fluidic agent 104 can be maintained, by the magnetic field F, at the target area 106 of the subject S. The user computer 114 can be configured to operate in conjunction with the controller 112 and magnetic source device 110 as an image-based control system, by which internal views of the body are visually displayed to a user to show the location of the fluidic agent 104 within the spinal region R. Imaging may be performed using a contrast agent included in the fluidic agent or separate from it, such as a fluorescence agent for use in fluorescence spectroscopy.

By visually monitoring the location of the fluidic agent 104 in real time, a user such as a medical professional may be enabled to specifically position or otherwise regulate one or more of the magnetic sources of the device 110. The user computer 114 may, for example, display a visual representation of the fluidic agent 104 as it travels through the spinal canal of the subject S to the target area 106, such that a user can adjust the separation distance of the device 110 from the spinal region R or otherwise regulate the magnetic field strength in order to optimize results. This may accommodate subjects across a large range of body sizes. For example, the device 110 may be positioned at a distance ranging between 0 and about 10 cm from the spinal region R, depending on the anatomy of the particular subject S. The magnetic source device 110 can be configured to produce a magnetic field F having specific parameters that may be established based on the separation distance between the device 110 and the subject S. For example, for a separation distance ranging from 0 to about 10 cm, the magnetic source device 110 can be configured to produce axial fields ranging from 0 to about 1000 gauss and gradients from 0 to about 50 gauss/mm

Although not specifically shown in FIG. 1, according to an alternative embodiment, one or more electromagnetic patches may be placed at or on an external portion of the subject S, and may be pressed onto the skin of a human subject proximate the spinal region R. For example, a pad-type arrangement with multiple electromagnets distributed in a linear array or matrix may be used, wherein each one of the electromagnets can be individually controlled. Such a configuration may allow for one or more specific areas of the spinal region R to be selectively magnetized, which may thereby determine and control the respective location in which the fluidic agent 104 can be maintained or towards or away from which it can be steered magnetically (i.e. moved). This configuration may also allow for the fluidic agent 104 to be guided through the spinal canal to the target area 106 from a different location.

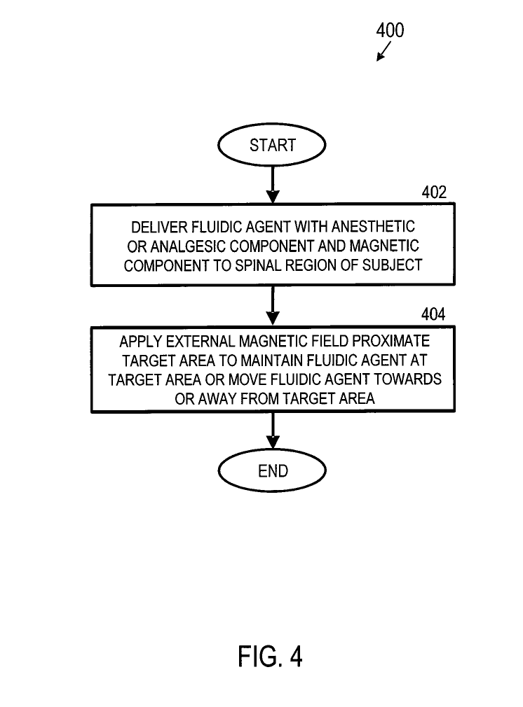

FIG. 4 illustrates operational steps of a method 400 for magnetic control of a fluidic agent in a spinal region of a subject, according to an exemplary embodiment of the present invention. The method 400 begins at block 402, where a fluidic agent is delivered to a spinal region of a subject, and more particularly an intrathecal space. The fluidic agent includes an anesthetic or analgesic agent and a magnetic agent. At block 404, a magnetic field is applied to an area proximate a target area of the spinal region such as to maintain the fluidic agent at the target area, move the fluidic agent towards the target area, or move the fluidic agent away from the target area.

The magnetic field can be applied from a location that is external to the spinal region of the subject. The target area can correspond to an intrathecal space of the spinal canal of the subject, and in particular one or more particular vertebral levels. In an exemplary embodiment, the magnetic field is to be applied such as to move the fluidic agent away from an area proximate the T1-T4 vertebral levels, in order to minimize anesthesia of cardioaccelerator fibers of the subject. In another exemplary embodiment, the magnetic field is applied such as to move the fluidic agent towards the T4 vertebral level, for anesthesia of the upper abdomen of the subject (in an abdominal surgery or Caesarean section procedure, for instance). In yet another exemplary embodiment, the magnetic field is applied such as to move the fluidic agent towards an area proximate the T6-T7 vertebral levels, for anesthesia of the lower abdomen of the subject (in an appendectomy, for instance).

In yet another exemplary embodiment, the magnetic field is applied such as to move the fluidic agent towards an area proximate the T10 vertebral level, for anesthesia of the pelvis of the subject (in hip surgery, prostate surgery, or vaginal delivery, for instance). In yet another exemplary embodiment, the magnetic field is applied such as to move the fluidic agent towards an area proximate the L1-L3 vertebral levels, for anesthesia of at least one lower extremity of the subject. In yet another exemplary embodiment, the magnetic field is applied such as to move the fluidic agent towards the L2-L3 vertebral level, for anesthesia of a foot of the subject. In yet another exemplary embodiment, the magnetic field is applied such as to move the fluidic agent towards the S2-S5 vertebral levels, for anesthesia of the perineum (in a hemorrhoidectomy, for instance).

Width of distribution of the fluidic agent 104 may be controlled with respect to particular areas by employing magnets of various sizes. For example, for a narrow width of distribution with respect to vertebral levels T6-T7, a small, single magnet may be applied to a focused spot, and for a wide width of distribution with respect to vertebral levels T6-12, a series of small magnets may be applied to multiple spots, or a single, large magnet may be applied to the area.

The anesthetic component of the fluidic agent can include a local anesthetic and the magnetic component can include a ferrofluid. The magnetic agent can include a suspension of particles, where each of the particles have a permanent magnetic moment or induced magnetic moment. The suspension of particles can include particles with permanent magnetic moments and induced magnetic moments. The magnetic field can be applied from a source comprising at least one electromagnet and can be a static magnetic field from diamagnetic, paramagnetic, and/or ferromagnetic source, and can have an axial field in a range between 0 and about 1000 gauss and, more particularly, about 135 gauss. The magnetic field can have a gradient in a range between 0 and about 50 gauss/mm and, more particularly, about 5 gauss/mm.

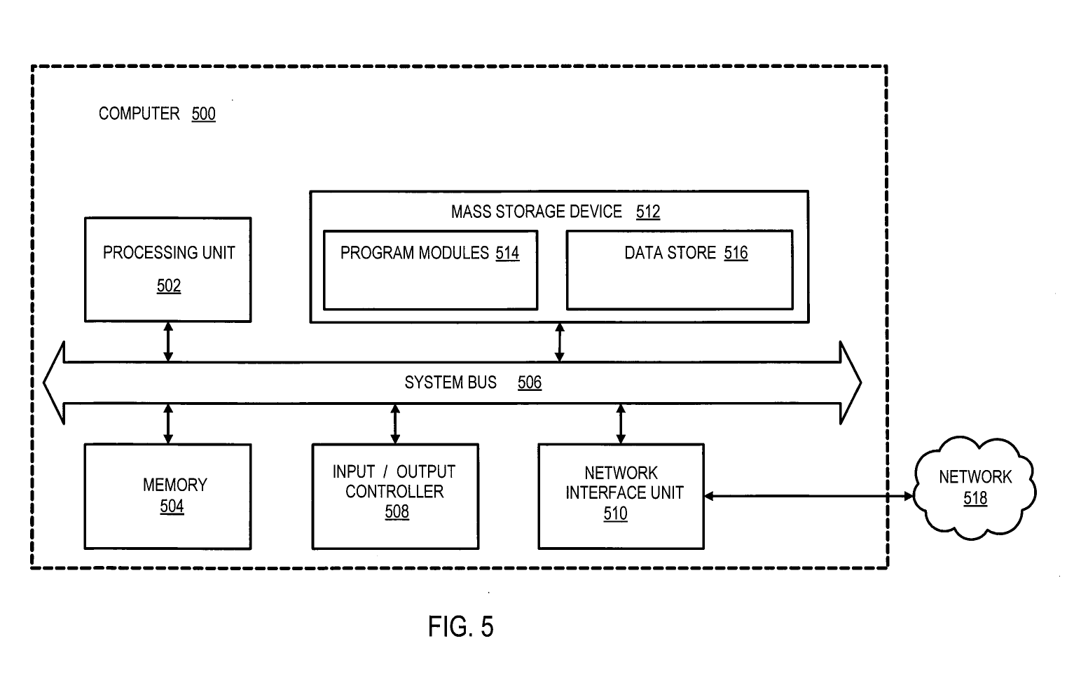

FIG. 5 is a computer architecture diagram showing illustrative computer hardware architecture for a computer 500 capable of implementing aspects of the present invention according to exemplary embodiments disclosed herein. As an exemplary implementation, a computer 500 may include one or more of the components shown in FIG. 1A. For example, the computer 500 may be configured to function as the user computer 114 operatively coupled to the controller 112 shown in FIG. 1A and described above. The computer 500 may be configured to perform one or more functions associated with exemplary embodiments illustrated in FIGS. 1B, FIGS. 3A-3D, and FIG. 4.

The computer 500 includes a processing unit 502, a system memory 504, and a system bus 506 that couples the memory 504 to the processing unit 502. The computer 500 further includes a mass storage device 512 for storing computer-executable program modules 514. The program modules 514 may include computer-executable software applications for performing real-time visual monitoring and control functions described above with reference to FIG. 1A. The mass storage device 512 further includes a data store 516. The mass storage device 512 is connected to the processing unit 502 through a mass storage controller (not shown) connected to the bus 506. The mass storage device 512 and its associated computer-storage media provide non-volatile storage for the computer 500. Although the description of computer-storage media contained herein refers to a mass storage device, such as a hard disk or CD-ROM drive, it should be appreciated by those skilled in the art that computer-storage media can be any available computer storage media that can be accessed by the computer 500.

By way of example, and not limitation, computer-storage media may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 500.

According to various embodiments, the computer 500 may operate in a networked environment using logical connections to remote computers through a network 518. The computer 500 may connect to the network 518 through a network interface unit 510 connected to the bus 506. It should be appreciated that the network interface unit 510 may also be utilized to connect to other types of networks and remote computer systems. The computer 500 may also include an input/output controller 508 for receiving and processing input from a number of input devices. The bus 506 may enable the processing unit 502 to read code and/or data to/from the mass storage device 512 or other computer-storage media. The computer-storage media may represent apparatus in the form of storage elements that are implemented using any suitable technology, including but not limited to semiconductors, magnetic materials, optics, or the like.

Computer storage media may include volatile and non-volatile, removable and non-removable media implemented in any method or technology for the non-transitory storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. Computer storage media does not include transitory signals.

The program module 514 may include software instructions that, when loaded into the processing unit 502 and executed, cause the computer 500 to provide functions for magnetic control of an anesthetic or other therapeutic or diagnostic agent. The program modules 514 may also provide various tools or techniques by which the computer 500 may participate within the overall systems or operating environments using the components, flows, and data structures discussed throughout this description. In general, the program module 514 may, when loaded into the processing unit 502 and executed, transform the processing unit 502 and the overall computer 500 from a general-purpose computing system into a special-purpose computing system. The processing unit 502 may be constructed from any number of transistors or other discrete circuit elements, which may individually or collectively assume any number of states. More specifically, the processing unit 502 may operate as a finite-state machine, in response to executable instructions contained within the program modules 514. These computer-executable instructions may transform the processing unit 502 by specifying how the processing unit 502 transitions between states, thereby transforming the transistors or other discrete hardware elements constituting the processing unit 502.

Encoding the program module 514 may also transform the physical structure of the computer-storage media. The specific transformation of physical structure may depend on various factors, in different implementations of this description. Examples of such factors may include, but are not limited to: the technology used to implement the computer-storage media, whether the computer storage media are characterized as primary or secondary storage, and the like. For example, if the computer-storage media are implemented as semiconductor-based memory, the program modules 514 may transform the physical state of the semiconductor memory, when the software is encoded therein. For example, the program modules 514 may transform the state of transistors, capacitors, or other discrete circuit elements constituting the semiconductor memory.

As another example, the computer-storage media may be implemented using magnetic or optical technology. In such implementations, the program modules 514 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

### EXEMPLARY IMPLEMENTATIONS AND RESULTS

The following describes examples of practicing concepts and technologies presented herein, and corresponding results, in accordance with aspects of the present invention.

### EXAMPLE 1

Methods and Related System

A model of the spine was constructed using standard, clear, polyvinyl-chloride tubing 204 (internal diameter 1.25″) as the surrogate spinal canal and 0.9% sodium chloride as mock cerebrospinal fluid. Two local anesthetic solutions were developed. The first solution (non-magnetic) consisted of equal parts hyperbaric lidocaine (5% lidocaine in 7.5% dextrose) and methylene blue (10 mg/mL). The two agents were combined in two 10 cc syringes connected by a standard stopcock and agitated manually. The calculated specific gravity of this solution was 1.02. The second solution (magnetic) consisted of equal parts hyperbaric lidocaine (5% lidocaine in 7.5% dextrose), methylene blue (10 mg/mL), and a water-based ferrofluid (EMG 700, Ferrotec Corporation, Santa Clara, Calif.). The three agents were combined in two 10 cc syringes connected by a standard stopcock and agitated manually. The calculated specific gravity of this solution (collectively represented as 206) was 1.07.

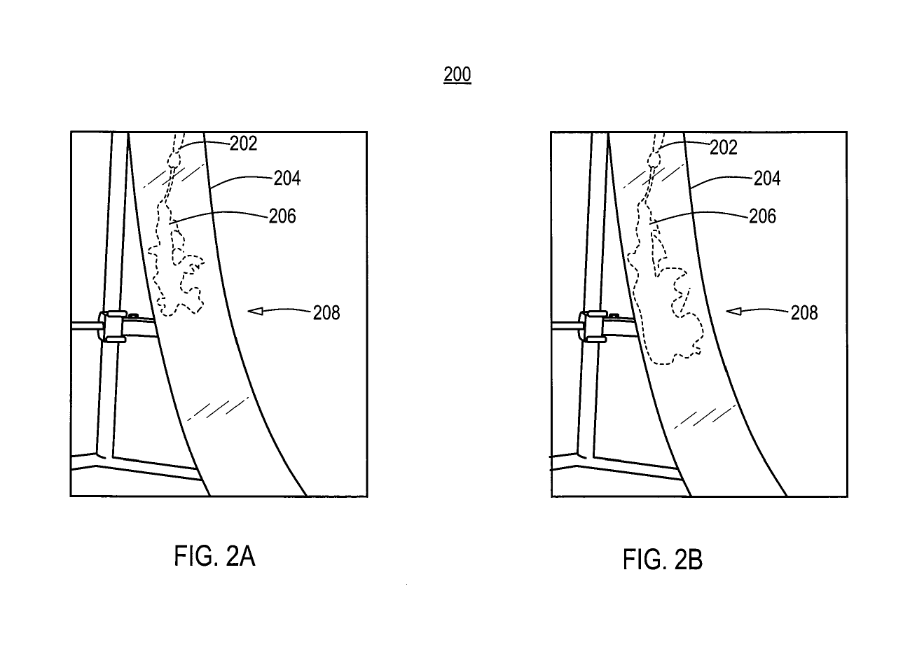

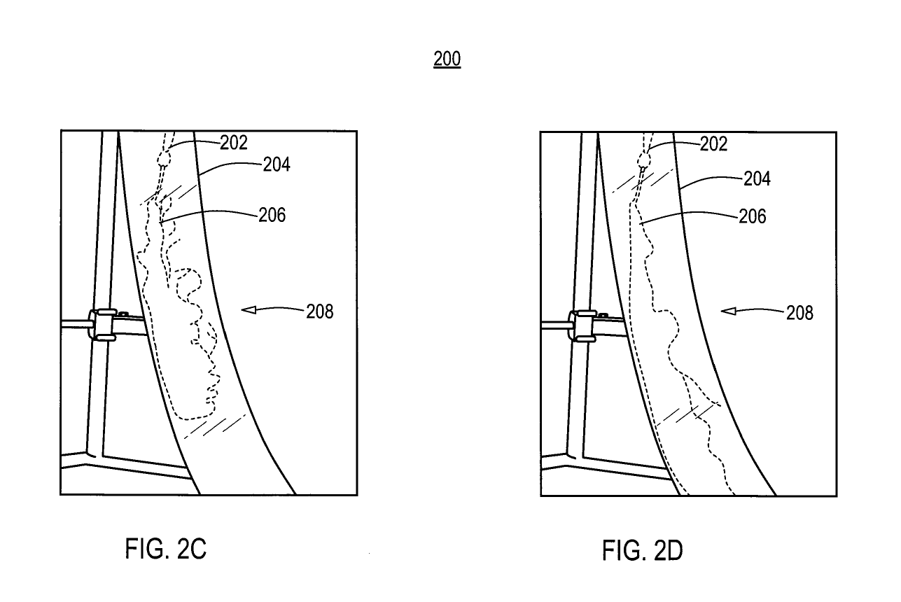

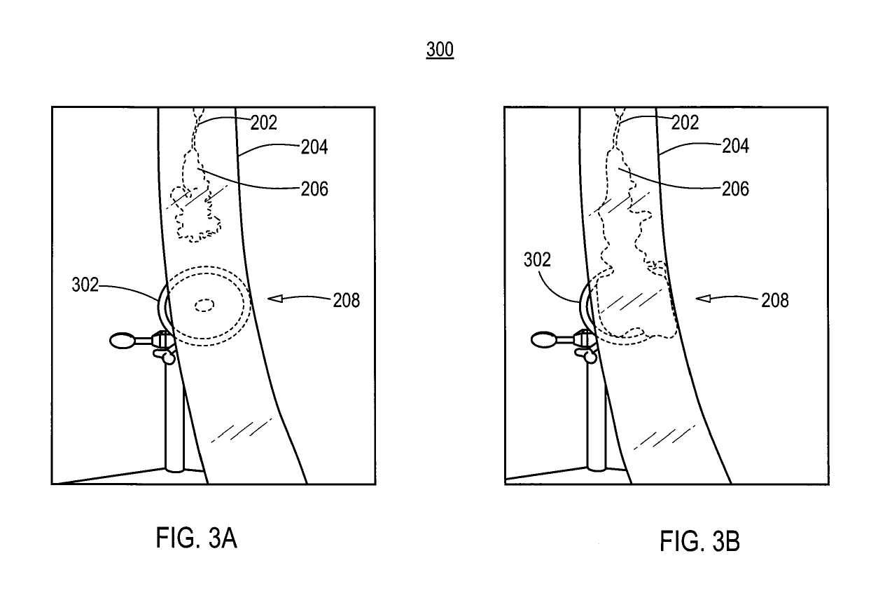

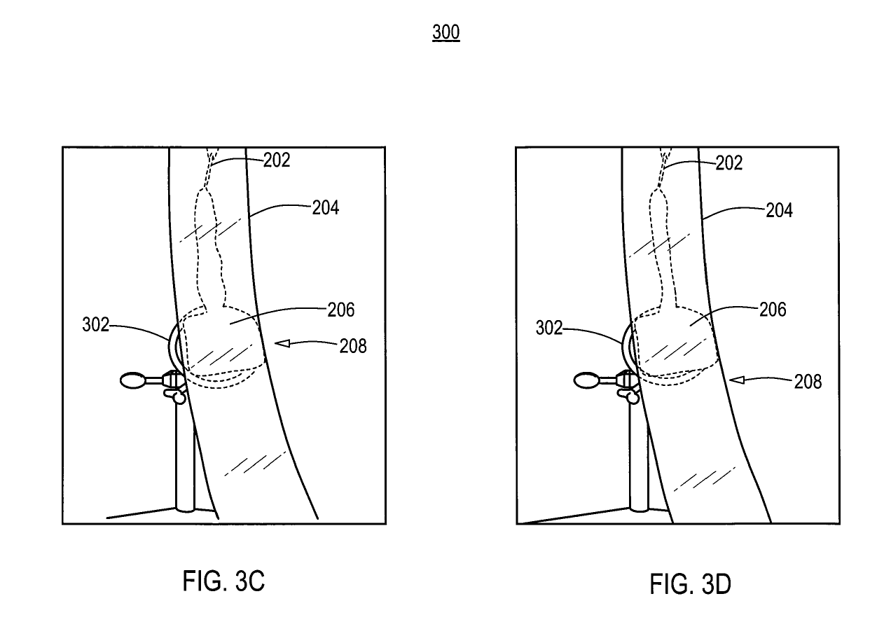

A 16-gauge introducer needle was placed or inserted into the inner curvature of the model spine, through which a 22-gauge pencil point needle 202 was placed. A permanent magnet 302 was placed underneath the needle 202 in an area 208 where the angle between the spine model and the earth's surface was approximately 45 degrees (and the cosine of the angle between the gravitational vector and the model was 0.707). The axial and lateral fields and gradients in the tube 204 (approximately 45 mm above the magnet 302) were approximately 135 and 50 gauss and approximately 5 gauss/mm and 2 gauss/mm, respectively. One mL of both agents was injected, slowly, by hand, over approximately one minute, with (FIGS. 2A-2D) and without (FIGS. 3A-3D) the magnet 302 in place. Fluid movement was captured by a digital camera operating in video mode.

To confirm that the methylene blue completely and irreversibly mixed with the local anesthetic and ferrofluid, both the magnetic and non-magnetic solutions were centrifuged at 6,000 RPM for 2 minutes. The color appeared to be uniform throughout the solution, and consistent with a control.

Results

The magnetic field had no effect on the movement of the non-magnetic fluid, which rapidly settled in the dependent region of the spine model (FIGS. 2A-2D). By contrast, it prevented gravitationally-dependent settling of the magnetic solution (FIGS. 3A-3D). Subsequent movement of the magnet against the gravitational vector caused the solution to move in opposition to gravity.

Centrifugation of the non-magnetic and magnetic solutions revealed no discernable separation, suggesting that the methylene blue completely and irreversibly mixed with the local anesthetic and ferrofluid.

Discussion

Incorporation of a ferrofluid into a local anesthetic solution, combined with application of an external magnetic field in an in vitro spine model, allowed control of position of a solution of ferrofluid, dye, and local anesthetic against gravity, suggesting an additional mechanism by which anesthesia providers may prevent high spinal block. As described above, the “high spinal” is a feared, potentially fatal complication of subarachnoid blockade in obstetric anesthesia. ([1], [2]). Neither the volume nor concentration of anesthetic agent appears to affect block height. The above-mentioned results from implementing methods disclosed herein in accordance with the exemplary embodiment shown in FIGS. 3A-3D demonstrate that application of a weak magnetic field to a local anesthetic solution containing nanoparticles with a fixed magnetic moment can overcome the effects of gravity on such a solution, and that movement of the magnetic field allows for manipulation of the solution. Potential uses of this technology also include facilitating one-sided blocks (e.g. hip surgery) and targeting specific dermatomal levels that are not gravitationally-dependent.

Although the ferrofluid used here is not yet FDA-approved for use with human subjects, magnetic particles such as magnetite have been used in a variety of biological settings, including thermal ablation of tumors and drug targeting in thrombolytic therapy. ([4]-[7]). The centrifuge test suggests that the methylene blue completely and irreversibly mixed with the local anesthetic and ferrofluid. The magnetic fields and gradients utilized in this test (i.e. Example 1) were relatively weak. Use of an electromagnet would allow a medical practitioner to apply any appropriate level of strength-controlled and directionally targeted magnetic field. ([8]). Uses of this technology include facilitating one-sided blocks (e.g. hip surgery), targeting specific dermatomal levels that are not gravitationally-dependent, and attenuation of the “high” spinal.

Numerous characteristics and advantages have been set forth in the foregoing description, together with details of structure and function. While the present invention has been disclosed in several forms, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

LIST OF REFERENCES

[1] Lee L A, Posner K L, Domino K B, Caplan R A, Cheney F W. Injuries associated with regional anesthesia in the 1980s and 1990s: a closed claims analysis. Anesthesiology 2004;101:143-52.

[2] Auroy Y, Narchi P, Messiah A, Litt L, Rouvier B, Samii K. Serious complications related to regional anesthesia: results of a prospective survey in France. Anesthesiology 1997;87:479-86.

[3] Van Zundert A A, Grouls R J, Korsten H H, Lambert D H. Spinal anesthesia. Volume or concentration—what matters? Reg Anesth 1996;21:112-8.

[4] Kawashita M, Tanaka M, Kokubo T, Inoue Y, Yao T, Hamada S, Shinjo T. Preparation of ferrimagnetic magnetite microspheres for in situ hyperthermic treatment of cancer. Biomaterials 2005;26:2231-8.

[5] Kempe M, Kempe H, Snowball I, Wallen R, Arza C R, Gotberg M, Olsson T. The use of magnetite nanoparticles for implant-assisted magnetic drug targeting in thrombolytic therapy. Biomaterials 2010;31:9499-510.

[6] Grady M S, Howard M A, 3rd, Broaddus W C, Molloy J A, Ritter R C, Quate E G, Gillies G T. Magnetic stereotaxis: a technique to deliver stereotactic hyperthermia. Neurosurgery 1990;27:1010-5; discussion 5-6.

[7] Schwagten B, Jordaens L, Rivero-Ayerza M, Van Belle Y, Knops P, Thornton I A, Szili-Torok T. A randomized comparison of transseptal and transaortic approaches for magnetically guided ablation of left-sided accessory pathways. Pacing Clin Electrophysiol 2010;33:1298-303.

[8] Gillies G T, Ritter R C, Broaddus W C, Grady M S, Howard III M A, McNeil R G. Magnetic manipulation instrumentation for medical physics research. Review of Scientific Instruments 1994;65:533-62.

[9] U.S. Patent Application Publication No. US2012/0029167 A1, Ishikawa, et al., “Auto Magnetic Metal Salen Complex Compound”, Feb. 2, 2012.

What is claimed is:

1. A method, comprising:
delivering a fluidic agent to a spinal region of a subject, the fluidic agent comprising at least one of an anesthetic and analgesic component, and a magnetic component; and
applying a magnetic field to an area proximate a target area of the spinal region such as to maintain the fluidic agent at the target area, move the fluidic agent towards the target area, or move the fluidic agent away from the target area.

2. The method of claim 1, wherein the magnetic field is applied from a location that is external to the spinal region of the subject.

3. The method of claim 1, wherein the target area corresponds to an intrathecal space of the spinal canal of the subject.

4. The method of claim 1, wherein the target area corresponds to one or more particular vertebral levels.

5. The method of claim 1, wherein applying the magnetic field to the area proximate the target area of the spinal region comprises causing the fluidic agent to move away from an area proximate the T1-T4 vertebral levels such as to minimize anesthesia of cardioaccelerator fibers of the subject.

6. The method of claim 1, wherein applying the magnetic field to the area proximate the target area of the spinal region comprises causing the fluidic agent to move towards the T4 vertebral level such as to provide for anesthesia of the upper abdomen of the subject.

7. The method of claim 1, wherein applying the magnetic field to the area proximate the target area of the spinal region comprises causing the fluidic agent to move towards an area proximate the T6-T7 vertebral levels such as to provide for anesthesia of the lower abdomen of the subject.

8. The method of claim 1, wherein applying the magnetic field to the area proximate the target area of the spinal region comprises causing the fluidic agent to move towards an area proximate the T10 vertebral level such as to provide for anesthesia of the pelvis of the subject.

9. The method of claim 1, wherein applying the magnetic field to the area proximate the target area of the spinal region comprises causing the fluidic agent to move towards an area proximate the L1-L3 vertebral levels such as to allow for anesthesia of at least one lower extremity of the subject.

10. The method of claim 1, wherein applying the magnetic field to the area proximate the target area of the spinal region comprises causing the fluidic agent to move towards the L2-L3 vertebral level such as to provide for anesthesia of a foot of the subject.

11. The method of claim 1, wherein applying the magnetic field to the area proximate the target area of the spinal region comprises causing the fluidic agent to move towards the S2-S5 vertebral levels such as to provide for anesthesia of the perineum.

12. The method of claim 1, wherein the anesthetic agent comprises a local anesthetic agent.

13. The method of claim 1, wherein the magnetic agent comprises a ferrofluid.

14. The method of claim 1, wherein the magnetic agent comprises a suspension of particles, each particle having a permanent magnetic moment.

15. The method of claim 1, wherein the magnetic agent comprises a suspension of particles, each particle having an induced magnetic moment.

16. The method of claim 1, wherein the magnetic agent comprises a suspension of particles having permanent magnetic moments and induced magnetic moments.

17. The method of claim 1, wherein the magnetic field is applied from a source comprising at least one electromagnet.

18. The method of claim 1, wherein the magnetic field is a static magnetic field from a diamagnetic source.

19. The method of claim 1, wherein the magnetic field is a static magnetic field from a paramagnetic source.

20. The method of claim 1, wherein the magnetic field is a static magnetic field from a ferromagnetic source.

21. The method of claim 1, wherein magnetic field comprises an axial field in a range between 0 and about 1000 gauss.

22. The method of claim 1, wherein the magnetic field comprises an axial field of about 135 gauss.

23. The method of claim 1, wherein the magnetic field comprises a gradient in a range between 0 and about 50 gauss/mm.

24. The method of claim 1, wherein the magnetic field comprises a gradient of about 5 gauss/mm.

25. A system, comprising:
a fluid delivery device configured to deliver a fluidic agent to a spinal region of a subject, the fluidic agent comprising at least one of an anesthetic and analgesic component, and a magnetic component; and
at least one magnet configured to apply a magnetic field to an area proximate a target area of the spinal region such as to maintain the fluidic agent at the target area, move the fluidic agent towards the target area, or move the fluidic agent away from the target area.

26. The system of claim 25, further comprising a programmable controller that is coupled to the at least one magnet and configured to control at least one of the field strength and gradient of the magnetic field.

27. The system of claim 25, wherein the at least one magnet is configured to apply the magnetic field from a location that is external to the spinal region of the subject.

28. The system of claim 25, wherein the target area corresponds to an intrathecal space of the spinal canal of the subject.

29. The system of claim 25, wherein the target area corresponds to one or more particular vertebral levels.

30. The system of claim 25, wherein the at least one magnet is configured to apply the magnetic field such as to cause the fluidic agent to move away from an area proximate the T1-T4 vertebral levels and such as to minimize anesthesia of cardioaccelerator fibers of the subject.

31. The system of claim 25, wherein the at least one magnet is configured to apply the magnetic field such as to cause the fluidic agent to move towards the T4 vertebral level and such as to provide for anesthesia of the upper abdomen of the subject.

32. The system of claim 25, wherein the at least one magnet is configured to apply the magnetic field such as to cause the fluidic agent to move towards an area proximate the T6-T7 vertebral levels and such as to provide for anesthesia of the lower abdomen of the subject.

33. The system of claim 25, wherein the at least one magnet is configured to apply the magnetic field such as to cause the fluidic agent to move towards an area proximate the T10 vertebral level and such as to provide for anesthesia of the pelvis of the subject.

34. The system of claim 25, wherein the at least one magnet is configured to apply the magnetic field such as to cause the fluidic agent to move towards an area proximate the L1-L3 vertebral levels and such as to allow for anesthesia of at least one lower extremity of the subject.

35. The system of claim 25, wherein the at least one magnet is configured to apply the magnetic field such as to cause the fluidic agent to move towards the L2-L3 vertebral level and such as to provide for anesthesia of a foot of the subject.

36. The system of claim 25, wherein the at least one magnet is configured to apply the magnetic field such as to move towards the S2-S5 vertebral levels and such as to provide for anesthesia of the perineum.

37. The system of claim 25, wherein the fluidic agent comprises a local anesthetic agent.

38. The system of claim 25, wherein the magnetic agent comprises a ferrofluid.

39. The system of claim 25, wherein the magnetic agent comprises a suspension of particles, each particle having a permanent magnetic moment.

40. The system of claim 25, wherein the magnetic agent comprises a suspension of particles, each particle having an induced magnetic moment.

41. The system of claim 25, wherein the magnetic agent comprises a suspension of particles having permanent magnetic moments and induced magnetic moments.

42. The system of claim 25, wherein the at least one magnet comprises at least one electromagnet.

43. The system of claim 25, wherein the at least one magnet comprises at least one diamagnetic source.

44. The system of claim 25, wherein the at least one magnet comprises at least one paramagnetic source.

45. The system of claim 25, wherein the at least one magnet comprises at least one ferromagnetic source.

46. The system of claim 25, wherein the at least one magnet is configured to produce an axial field in a range between 0 and about 1000 gauss.

47. The system of claim 25, wherein the at least one magnet is configured to produce an axial field of about 135 gauss.

48. The system of claim 25, wherein the magnetic field comprises a gradient in a range between 0 and about 50 gauss/mm.

49. The system of claim 25, wherein the magnetic field comprises a gradient of about 5 gauss/mm.

50. A computer-readable storage medium having stored instructions that, when executed by one or more processors, cause a computer to perform functions that comprise
causing at least one magnet to apply a magnetic field to an area that is proximate a target area of a spinal region of a subject, such as to
maintain a fluidic agent at the target area,
move the fluidic agent towards the target area, or
move the fluidic agent away from the target area,
wherein the fluidic agent comprises at least one of an anesthetic component and an analgesic component, and a magnetic component.
